SEQUENCE LISTING (13 sequences)

SEQ ID NO: 1 - protein, 5 aa, synthetic construct
SYVMH

SEQ ID NO: 2 - protein, 6 aa, synthetic construct
NPYNDG

SEQ ID NO: 3 - protein, 12 aa, synthetic construct
GTYYYGTRVFDY

SEQ ID NO: 4 - protein, 16 aa, synthetic construct
RSSKSLQNVNGNTYLY

SEQ ID NO: 5 - protein, 7 aa, synthetic construct
RMSNLNS

SEQ ID NO: 6 - protein, 9 aa, synthetic construct
MQHLEYPIT

SEQ ID NO: 7 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 8 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 9 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 107 aa, synthetic construct [sequence omitted]

SEQ ID NO: 11 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 556 aa, Homo sapiens [sequence omitted]

(19) United States
(12) Patent Application Publication
(10) Pub. No.: US 2026/0345771 A1
(43) Pub. Date: Nov. 5, 2026

(54) THERAPY COMPRISING ANTI-CD19 ANTIBODY AND EZH2 MODULATORS

(71) Applicant: Incyte Corporation, Wilmington, DE (US)

(72) Inventors: Eveline Schaadt, Oberhaching (DE); Christina Heitmüller, Wolfratshausen (DE); Archana Reddy, Milton, MA (US)

(21) Appl. No.: 19/104,071

(22) PCT Filed: Aug. 16, 2023
(86) PCT No.: PCT/EP2023/072620

§ 371 Date: Feb. 14, 2025

Related U.S. Application Data
(60) Provisional application No. 63/398,634, filed on Aug. 17, 2022.
(60) Provisional application No. 63/417,812, filed on Oct. 20, 2022.

(51) Int. Cl. A61K 39/395; A61K 31/443; A61P 35/00; C07K 16/28

(52) U.S. Cl. CPC A61K 39/3955; A61K 31/443; A61P 35/00; C07K 16/2803

(57) ABSTRACT

The present disclosure provides methods, pharmaceutical compositions, and kits for treating cancer in patients in need thereof. The methods comprise administering to a patient in need a EZH2 modulator, such as Compound I, or a pharmaceutically acceptable salt thereof, in combination with one or more anti-CD 19 antibodies. Also provided are medicaments for use in treating cancer.

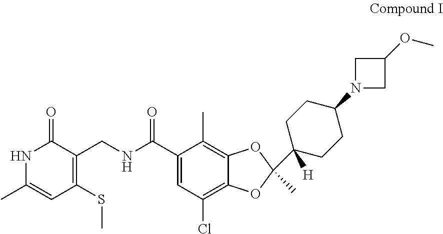

### FIELD

The present disclosure is directed to a combination comprising an anti-CD19 antibody thereof and an EZH2 modulator for use in the treatment of various cancers.

### BACKGROUND

Non-Hodgkin's lymphoma (NHL) is among the most common cancers in the United States and Europe with more than 70,000 and 93,000 new cases diagnosed every year, respectively. Siegel R. L., et al., CA Cancer J. Clin. 68 (1): 7-30 (2018); Ferlay J., et al., Eur. J. Cancer 103:356-87 (2018). NHL is a heterogeneous group of malignancies with varying clinical characteristics that are optimally managed through a range of different treatment modalities. The spectrum of NHL includes more indolent variants such as follicular and marginal zone lymphomas, to more aggressive subtypes such as diffuse large B-cell lymphoma (DLBCL) and Burkitt lymphoma (BL). While systemic chemotherapy is a mainstay of treatment for most NHL variants, antitumor directed monoclonal antibodies have an important role in the treatment of this disease. Oflazoglu E., et al., MAbs 2 (1): 14-9 (2010). Monoclonal antibodies such as rituximab, which targets the B-cell antigen CD20, are part of the standard treatment regimens for many B-cell NHLs. Keating G M, Drugs 70 (11): 1445-76 (2010). However, once NHL becomes refractory to standard chemotherapy and antibody-based therapies, the overall prognosis is poor, with limited long-term survival. Thus, novel and effective therapies are needed to address this high unmet medical need.

Indolent NHL (iNHL) represents 40% of all NHL subtypes, with follicular lymphoma occurring with the greatest frequency. Harris N. L., et al., Ann. Oncol. 10 (12): 1419-32 (1999). iNHL presents with a broad spectrum of disease characteristics. Patients often experience a chronic relapsing and remitting disease course and are exposed to several successive treatment regimens, resulting eventually in death due to disease progression. In general, treatment is reserved for patients who develop significant symptoms or who are sufficiently high risk to merit early therapy. Gribben J. G., Blood 109 (11): 4617-26 (2007).

For patients with iNHL who initially respond (complete or partial response (PR) with a time to progression of at least 6 months) and then experience relapse after single-agent rituximab, retreatment with either rituximab alone or in combination with chemotherapy is frequently given. Gribben J. G., Blood 109 (11): 4617-26 (2007); Kahl B. S., et al., J. Clin. Oncol. 32 (28): 3096-102 (2014); NCCN Clinical Practice Guidelines in Oncology (NCCN Guidelines): B-Cell Lymphomas (Version 3.2019), National Comprehensive Cancer Network, May 6, 2019. Patients who become refractory to rituximab alone or in combination with chemotherapy have limited options for effective treatment.

Aggressive Non-Hodgkin's Lymphoma (aNHL) accounts for approximately 30 to 40% of all NHL (Project TN-HsLC, Blood 89 (11): 3909-18 (1997)) and DLBCL is the most common histological subtype. Beham-Schmid C., Aggressive lymphoma 2016: revision of the WHO classification, Memo 10 (4): 248-54 (2017). Combination chemotherapy with the addition of rituximab is standard of care for patients with newly diagnosed DLBCL. However, approximately 40% of patients with DLBCL relapse following initial immunochemotherapy. Vaidya R., et al., Ann. Oncol. 25 (11): 2124-33 (2014). For eligible patients, salvage chemotherapy regimens followed by autologous stem cell transplantation is the standard of care. However, many patients are not eligible for transplantation due to age and other medical co-morbidities. While various salvage regimens comprising combination chemotherapy are available for relapsed/refractory disease, no standard salvage regimen exists currently. There remains an unmet need for NHL patients who have early relapses or who are not responsive to anti-CD20 treatment regimens.

CD19 is a 95-kDa transmembrane glycoprotein of the immunoglobulin superfamily containing two extracellular immunoglobulin-like domains and an extensive cytoplasmic tail. The protein is a pan-B lymphocyte surface receptor and is ubiquitously expressed from the earliest stages of pre-B cell development onwards until it is down-regulated during terminal differentiation into plasma cells. It is B-lymphocyte lineage specific and not expressed on hematopoietic stem cells and other immune cells, except some follicular dendritic cells. CD19 functions as a positive regulator of B cell receptor (BCR) signaling and is important for B cell activation and proliferation and in the development of humoral immune responses. It acts as a co-stimulatory molecule in conjunction with CD21 and CD81 and is critical for B cell responses to T-cell-dependent antigens. The cytoplasmic tail of CD19 is physically associated with a family of tyrosine kinases that trigger downstream signaling pathways via the src-family of protein tyrosine kinases. CD19 is an attractive target for cancers of lymphoid origin since it is highly expressed in nearly all-chronic lymphocytic leukemia (CLL) and non-Hodgkin's lymphomas (NHL), as well as many other different types of leukemias, including acute lymphocytic leukemia (ALL) and hairy cell leukemia (HCL).

Tafasitamab (former names: MOR208 and XmAb®5574) is a humanized monoclonal antibody that targets the antigen CD19, a transmembrane protein involved in B-cell receptor signaling. Tafasitamab has been engineered in the IgG Fc-region to enhance antibody-dependent cell-mediated cytotoxicity (ADCC), thus improving a key mechanism for tumor cell killing and offering potential for enhanced efficacy compared to conventional antibodies, i.e. non-enhanced antibodies. Tafasitamab has or is currently being studied in several clinical trials, such as in CLL, ALL and NHL. Tafasitamab received accelerated approval from the US Food and Drug Administration (FDA) in July 2020 for use in combination with lenalidomide to treat adults with R/R DLBCL. In August 2021, the European Commission granted conditional marketing authorization for tafasitamab in combination with lenalidomide, followed by tafasitamab monotherapy, for the treatment of adult patients with relapsed or refractory diffuse large B-cell lymphoma (DLBCL) who are not eligible for autologous stem cell transplantation (ASCT). Despite recent discoveries and developments of several anti-cancer agents, due to poor prognosis for many types of cancers including CD19-expressing tumors, there is still a need for an improved method or therapeutic approach for treating such types of cancers.

New combinations of therapeutic agents that provide a beneficial effect in the treatment of cancers are desirable in order to prolong patient's lives while maintaining a high quality of life. New combinations may provide an increased benefit as compared to each of the agents alone. In particular, combined treatment regimens may be helpful for patients suffering from disease conditions including proliferative disorders, and could potentially even decrease the rate of relapse or overcome the resistance to a particular anticancer agent sometimes seen in these patients. This is especially true in the case where the cancers may be resistant or refractory to currently available therapeutic regimens.

Thus, there is a need for new cancer treatment regimens, including combination therapies. The present inventors have discovered that combined administration of an antibody or antibody fragment specific for CD19 together with an EZH2 modulator has superior effects on the treatment of malignant lymphomas of B cell origin.

### SUMMARY

The present disclosure provides a novel combination for use in the treatment of a cancer, comprising an anti-CD19 antibody and an EZH2 modulator.

In one aspect, the present disclosure relates to methods of treating a cancer comprising administering to a human subject in need of said treatment an EZH2 modulator and an anti-CD19 antibody in combination.

In one aspect, the present disclosure relates to methods of treating a cancer comprising administering to a patient in need of said treating a combination of an EZH2 inhibitor, and an anti-CD19 antibody or antibody fragment.

In some embodiments, the anti-CD19 antibody comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising the sequence LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

In some embodiments, the anti-CD19 antibody comprises a heavy chain variable region comprising an HCDR1 region of SYVMH (SEQ ID NO: 1), an HCDR2 region of NPYNDG (SEQ ID NO: 2), and an HCDR3 region of GTYYYGTRVFDY (SEQ ID NO: 3) and a light chain variable region comprising an LCDR1 region of RSSKSLQNVNGNTYLY (SEQ ID NO: 4), an LCDR2 region of RMSNLNS (SEQ ID NO: 5), and an LCDR3 region of MQHLEYPIT (SEQ ID NO: 6).

In some embodiments, the anti-CD19 antibody comprises a heavy chain variable region of

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS
and
a light chain variable region of
(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

In some embodiments, the anti-CD19 antibody has effector function. In another aspect the antibody or antibody fragment specific for CD19 has an enhanced effector function. In one embodiment the effector function is ADCC. In one embodiment the antibody or antibody fragment specific for CD19 has an enhanced ADCC activity. In a further embodiment the antibody or antibody fragment specific for CD19 comprises an Fc domain comprising an amino acid substitution at position S239 and/or 1332, wherein the numbering is according to the EU index as in Kabat. In a further embodiment the antibody or antibody fragment specific for CD19 comprises an Fc domain comprising an S239D amino acid substitution and an I332E amino acid substitution, wherein the numbering is according to the EU index as in Kabat.

In some embodiments, the anti-CD19 antibody comprises a heavy chain constant region of

(SEQ ID NO: 9)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPDVFLFPPKPKDTLMISRTPEVTCVVVDVS
HEDPEVQFNWYVDGVEVHNAKTKPREEQFNSTFRVVSVLTVVHQDWLNGK
EYKCKVSNKALPAPEEKTISKTKGQPREPQVYTLPPSREEMTKNQVSLTC
LVKGFYPSDIAVEWESNGQPENNYKTTPPMLDSDGSFFLYSKLTVDKSRW
QQGNVFSCSVMHEALHNHYTQKSLSLSPGK.

In some embodiments, the anti-CD19 antibody comprises a light chain constant region of

(SEQ ID NO: 10)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In some embodiments, the anti-CD19 antibody comprises a heavy chain constant region of

(SEQ ID NO: 9)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPDVFLFPPKPKDTLMISRTPEVTCVVVDVS
HEDPEVQFNWYVDGVEVHNAKTKPREEQFNSTFRVVSVLTVVHQDWLNGK
EYKCKVSNKALPAPEEKTISKTKGQPREPQVYTLPPSREEMTKNQVSLTC
LVKGFYPSDIAVEWESNGQPENNYKTTPPMLDSDGSFFLYSKLTVDKSRW
QQGNVFSCSVMHEALHNHYTQKSLSLSPGK

and a light chain constant region of

(SEQ ID NO: 10)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In some embodiments, the anti-CD19 antibody comprises a heavy chain region of

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K
and
a light chain region of
(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

In some embodiments, the anti-CD19 antibody is tafasitamab.

In some embodiments, the EZH2 modulator is administered orally.

In some embodiments, the EZH2 modulator is administered intravenously. In some embodiments, the EZH2 modulator is administered subcutaneously.

In some embodiments, the EZH2 modulator is administered by intravenous infusion.

In some embodiments, the EZH2 modulator is an EZH2 inhibitor.

In some embodiments, the anti-CD19 antibody is administered intravenously.

In some embodiments, the anti-CD19 antibody is administered by intravenous infusion.

In some embodiments, the anti-CD19 antibody is administered by subcutaneous injection.

In some embodiments, the anti-CD19 antibody is administered subcutaneously.

In some embodiments, the cancer is a CD19 positive cancer.

In some embodiments, the cancer is a hematological malignancy.

In some embodiments, the cancer is a lymphoma or a leukemia.

In some embodiments, the cancer is a chronic lymphocytic leukemia or a non-Hodgkin's lymphoma.

In some embodiments, the cancer is a CD19 positive chronic lymphocytic leukemia or a CD19 positive non-Hodgkin's lymphoma.

In some embodiments, the cancer is a non-Hodgkin's lymphoma. In some embodiments the human subject suffers from relapsed or refractory non-Hodgkins lymphoma. In some embodiments the cancer is follicular lymphoma (FL), marginal zone lymphoma (MZL), mantle cell lymphoma (MCL), Diffuse large B-cell lymphoma (DLBCL), or Burkitt lymphoma.

In some embodiments, the EZH2 modulator is administered once every two weeks, once every week, twice a week, three times a week, or daily.

In some embodiments, the EZH2 modulator is administered twice a week.

In some embodiments, the EZH2 modulator is administered once every week.

In some embodiments, the EZH2 modulator is administered on days 1, 4, 8, and 11 of a 21 day cycle.

In some embodiments, the anti-CD19 antibody is administered once every two weeks, once every week, twice a week, three times a week, or daily.

In some embodiments, the anti-CD19 antibody is administered once every two weeks.

In some embodiments, the anti-CD19 antibody is administered once every week.

In some embodiments, the anti-CD19 antibody is administered once every four weeks.

In some embodiments, the anti-CD19 antibody is administered once every eight weeks.

In some embodiments, the anti-CD19 antibody is administered on Day 1 of a treatment cycle.

In some embodiments, the treatment cycle is 21 days or 28 days.

In some embodiments, the EZH2 modulator and the anti-CD19 antibody are administered simultaneously once every eight weeks, once every four weeks, once every two weeks, once every week, twice a week, three times a week, or daily.

In some embodiments, the EZH2 modulator and the anti-CD19 antibody are administered simultaneously on days 1, 4, 8, and 11 of a 21 day cycle

In some embodiments, EZH2 modulator is administered once every two weeks, once every week, twice a week, three times a week, daily, or on days 1, 4, 8, and 11 of a 21 day cycle; and the anti-CD19 antibody is separately administered once every eight weeks, once every four weeks, once every two weeks, once every week, twice a week, three times a week, or daily.

In one aspect, the present disclosure relates to a kit comprising a medicament for use in treating cancer in a subject in need of such treatment. The kit comprises a medicament comprising an EZH2 modulator, and instructions for administering the EZH2 modulator and the one or more anti-CD19 antibodies or antibody fragments; or the kit comprises a medicament comprising the one or more anti-CD19 antibodies or antibody fragments, and instructions for administering the one or more anti-CD19 antibodies or antibody fragments and an EZH2 modulator. The kit can contain both a medicament comprising an EZH2 modulator and a medicament comprising one or more anti-CD19 antibodies, and instructions for administering the EZH2 modulator and the one or more anti-CD19 antibodies. The kit can also comprise one or more additional therapeutic agents.

In one aspect, the present disclosure relates to a medicament for use in treating cancer in a subject in need of such treatment. The medicament comprises an EZH2 modulator and one or more anti-CD19 antibodies or antibody fragments. The medicament can also comprise one or more additional therapeutic agents.

### BRIEF DESCRIPTION OF THE FIGURES

FIG. 1: Viability assay. Effect of tafasitamab and Compound 1 alone and in combination on the viable cell count of different lymphoma cell lines as determined in CTG assay.

FIG. 2: Estimates of combinatorial effect according to linear mixed effect model. The error bars indicate the 95% confidence intervals.

FIG. 3: Viability assay. Dose titration curves of Compound 1 and tazemetostat in absence and presence of 0.1 nM tafasitamab in SU-DHL-4 (A) and SU-DHL-6 (B) cell lines.

FIG. 4: Upregulation of cell surface CD19 level by Compound 1 after 7 days of treatment.

FIG. 5: Tumor growth kinetics (mean±/−SEM) upon administration of different concentrations of Compound 1 and/or Tafasitamab.

FIG. 6: Mean tumor volumes of the individual treatment groups on day 38 after tumor implantation (day 21 after treatment start).

### DETAILED DESCRIPTION

### Definitions

To facilitate an understanding of the present disclosure, a number of abbreviations, terms, and phrases are defined below.

Unless defined otherwise, all technical and scientific terms used herein have the same meaning as is commonly understood by one of skill in the art to which this disclosure belongs. All patents and publications referred to herein are incorporated by reference in their entirety.

As used herein, the term “cancer” refers to a cellular disorder characterized by uncontrolled or dysregulated cell proliferation, decreased cellular differentiation, inappropriate ability to invade surrounding tissue, and/or ability to establish new growth at ectopic sites. The term “cancer” includes solid tumors and non-solid tumors, such as, for example, hematological tumors. The term “cancer” encompasses diseases of skin, tissues, organs, bone, cartilage, blood, and vessels. The term “cancer” further encompasses primary and metastatic cancers.

The term “CD19” refers to the protein known as CD19, having the following synonyms: B4, B-lymphocyte antigen CD19, B-lymphocyte surface antigen B4, CVID3, Differentiation antigen CD19, MGC12802, and T-cell surface antigen Leu-12. The term also encompasses naturally occurring variants of CD19, e.g., splice variants, allelic variants, and isoforms.

In an embodiment human CD19 has the amino acid sequence of:

(SEQ ID NO: 13)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR

“MOR208” and “XmAb 5574” and “tafasitamab” are used as synonyms for the anti-CD19 antibody according to Table 1. Table 1 provides the amino acid sequences of MOR208/tafasitamab. The MOR208 antibody is described in US patent U.S. Pat. No. 8,524,867, which is incorporated by reference in its entirety.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses polyclonal antibodies, monoclonal antibodies, antibody fragments (such as Fab, Fab′, F(ab′) 2, and Fv fragments), single chain Fv (scFv) mutants, multispecific antibodies such as bispecific antibodies for example generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology. The different classes of immunoglobulins have different and well-known subunit structures and three-dimensional configurations. Antibodies can be naked or conjugated to other molecules such as toxins, radioisotopes, etc.

The term “anti-CD19 antibody” or “an antibody that binds to CD19” refers to an antibody that is capable of binding CD19 with sufficient affinity such that the antibody is useful as a diagnostic and/or therapeutic agent in targeting CD19.

A “monoclonal antibody” refers to a homogeneous or substantially homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

As used herein, the term “effective amount” or “therapeutically effective amount” refers to an amount of a compound, or combination of one or more compounds that, when administered (either sequentially or simultaneously) elicits the desired biological or medicinal response, e.g., either destroys, slows or arrests the growth of the target cancer cells or slows or arrests the progression of the cancer in a patient. The therapeutically effective amount may vary depending upon the intended application (e.g., in vitro or in vivo), or the patient and disease condition being treated, and can depend on factors such as, e.g., the weight and age of the patient, the severity of the disease condition, the manner of administration and the like, which may readily be determined by one skilled in the art. The term “effective amount” or “therapeutically effective amount” also applies to an amount, such as one or more doses, that will induce a particular response in target cells, e.g., reduction of platelet adhesion and/or cell migration. For example, in some embodiments, the “therapeutically effective amount” as used herein refers to the amount of an EZH2 modulator and the amount of an anti-CD19 antibody that, when administered separately or in combination, have a beneficial effect. In some embodiments, the combined effect is additive. In some embodiments, the combined effect is synergistic. Further, it will be recognized by one skilled in the art that in the case of combination therapy, the amount of an EZH2 modulator and/or the amount of the anti-CD19 antibody may be used in a “sub-therapeutic amount”, i.e., less than the therapeutically effective amount of an EZH2 modulator or the anti-CD19 antibody alone.

In any form or composition, the administered dose(s) or the therapeutically effective (total) amount may be expressed as amount(s) of therapeutic substance(s) per patient as either based on (i) BSA, e.g., as mg/m², or (ii) amount, e.g., as mg.

The term “about” refers to approximately, in the region of, roughly, or around. When the term “about” is used in conjunction with a number or a numerical range, it means that the number or numerical range referred to is an approximation within experimental variability (or within statistical experimental error), and thus the number or numerical range may vary from, for example, between 1% and 15% of the stated number or numerical range. In general, the term “about” is used herein to modify a numerical value above and below the stated value by a variance of ±10%.

As used herein, “patient” generally means a mammal (e.g., human) who has been diagnosed with, exhibits symptoms of, or is otherwise believed to be afflicted with a disease, disorder, or condition (such as cancer). The term “patient” is used interchangeably herein with the term “subject”.

As used herein, “body surface area” (BSA) is calculated using a standard nomogram, e.g.,

$$BSA ( {m}^{2} ) = \sqrt{\frac{H t ( cm ) \times Wt ( kg )}{3600}} or BSA = \sqrt{\frac{Ht ( in ) \times Wt ( lb )}{3131}}$$

The terms “combination”, “pharmaceutical combination”, “combination administration”, “administered in combination,” and “administering a combination” refer to the administration of one therapy in addition to another therapy. Each component can be administered simultaneously or sequentially in any order at different points in time. As such, “in combination with” refers to administering of more than one pharmaceutically active ingredients (including, but not limited to, an EZH2 modulator and an anti-CD19 antibody as disclosed herein) to a patient. Combination administration may refer to simultaneous administration or may refer to sequential administration of an EZH2 modulator and an anti-CD19 antibody as disclosed herein.

The terms “simultaneous” and “simultaneously” refer to the administration of the an EZH2 modulator and an anti-CD19 antibody as disclosed herein, to a patient at the same time, or at two different time points that are separated by no more than about 2 hours. The simultaneous administration of an EZH2 modulator and an anti-CD19 antibody may be in a single dosage form or in separate dosage forms.

The terms “sequential” and “sequentially” refer to the administration of an EZH2 modulator and an anti-CD19 antibody as disclosed herein, to a patient at two different time points that are separated by more than about 2 hours, e.g., about 3 hours, about 4 hours, about 5 hours, about 8 hours, about 12 hours, 1 day, 2 days, 3 days, 4 days, 5 days, 6 days, 7 days or even longer.

The term “intermission” refers to a period that is subsequent to the administration of one or more particular pharmaceutically active ingredients to a patient in an intermittent regimen. Intermission refers to a rest period wherein a particular pharmaceutically active ingredient is not administered for at least one day.

The terms “synergy”, “synergism”, “synergistic” and “synergistic effect” which are used herein interchangeably refer to a situation where the combination of two or more agents produces a greater effect than the sum of the effects of each of the individual agents. The term encompasses a reduction in symptoms of the disorder to be treated, but also an improved side effect profile, improved tolerability, improved patient compliance, improved efficacy, or any other improved clinical outcome.

As used herein, the illustrative terms “include”, “such as”, “for example”, “e.g.” and the like (and variations thereof, e.g., “includes” and “including”, “examples”), unless otherwise specified, are intended to be non-limiting. That is, unless explicitly stated otherwise, such terms are intended to imply “but not limited to”, e.g., “including” means “including but not limited to”.

When used in connection to describe a chemical group that may have multiple points of attachment, a hyphen (-) designates the point of attachment of that group to the variable to which it is defined. For example, −S(C₃-C₇)cycloalkyl and —S[halo(C₁-C₄)alkyl] mean that the point of attachment for this group occurs on the sulfur atom.

The terms “halo” and “halogen” as used herein refer to an atom selected from fluorine (fluoro, —F), chlorine (chloro, —Cl), bromine (bromo, —Br), and iodine (iodo, —I).

The term “alkyl,” as used herein, refers to a monovalent saturated, straight- or branched-chain hydrocarbon radical, having unless otherwise specified, 1-10 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, and the like.

The term “haloalkyl” includes mono, poly, and perhaloalkyl groups where the halogens are independently selected from fluorine, chlorine, bromine, and iodine.

“Alkoxy” is an alkyl group which is attached to another moiety via an oxygen linker (—O(alkyl)). Non-limiting examples include methoxy, ethoxy, propoxy, and butoxy.

“Haloalkoxy” is a haloalkyl group which is attached to another moiety via an oxygen atom such as, e.g., but are not limited to —OCHCF₂ or —OCF₃.

The term “cycloalkyl” refers to a 3- to 12-membered (e.g., 3- to 7-membered) monocyclic, bicyclic (e.g., a bridged or spiro bicyclic ring), or polycyclic (e.g., tricyclic), hydrocarbon ring system that is completely saturated. Monocyclic cycloalkyl groups include, without limitation, cyclopropyl, cyclobutyl, cyclopentyl, cyclohexyl, cycloheptyl, and cyclooctyl. Bridged bicyclic cycloalkyl groups include, without limitation, bicyclo[3.2.1]octane, bicyclo[2.2.1]heptane, bicyclo[3.1.0]hexane, bicyclo[1.1.1]pentane, and the like. Spiro bicyclic cycloalkyl groups include, e.g., spiro[3.6]decane, spiro[4.5]decane, and the like. Fused cycloalkyl rings include, e.g., decahydronaphthalene, octahydropentalene, and the like. Where specified as being optionally substituted or substituted, substituents on a cycloalkyl (e.g., in the case of an optionally substituted cycloalkyl) may be present on any substitutable position and, include, e.g., the position at which the cycloalkyl group is attached.

The term “heterocyclyl” means a 3-12 membered (e.g., a 4-, 5-, 6- and 7-membered) saturated or partially unsaturated heterocyclic ring containing 1 to 4 heteroatoms independently selected from N, O, and S. It can be monocyclic, bicyclic (e.g., a bridged, fused, or spiro bicyclic ring), or tricyclic. The terms “heterocycle”, “heterocyclyl”, “heterocyclyl ring”, “heterocyclic group”, “heterocyclic moiety”, and “heterocyclic radical”, are used interchangeably herein. A heterocyclyl ring can be attached to its pendant group at any heteroatom or carbon atom that results in a stable structure. Examples of such saturated or partially unsaturated heterocyclic radicals include, without limitation, tetrahydrofuranyl, tetrahydrothienyl, tetrahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. A heterocyclyl group may be mono- or bicyclic. The term “heterocyclyl” also includes, e.g., unsaturated heterocyclic radicals fused to another unsaturated heterocyclic radical or aryl or heteroaryl ring, such as for example, tetrahydronaphthyridine, indolinone, dihydropyrrolotriazole, imidazopyrimidine, quinolinone, dioxaspirodecane. Where specified as being optionally substituted or substituted, substituents on a heterocyclyl (e.g., in the case of an optionally substituted heterocyclyl) may be present on any substitutable position and, include, e.g., the position at which the heterocyclyl group is attached.

The term “spiro” refers to two rings that shares one ring atom (e.g., carbon).

The term “fused” refers to two rings that share two adjacent ring atoms with one another.

The term “bridged” refers to two rings that share three or more ring atoms with one another.

Unless otherwise stated, structures depicted herein are meant to include chemical entities which differ only in the presence of one or more isotopically enriched atoms. For example, chemical entities having the present structure except for the replacement of a hydrogen atom by a deuterium or tritium, or the replacement of a carbon atom by a ¹³C- or ¹⁴C-enriched carbon are within the scope of the invention. In one aspect, all atoms on a compound described herein (e.g., a disclosed EZH2 modulator) are present in natural abundance.

Unless stereochemical configuration is denoted, structures depicted herein are meant to include all stereochemical forms of the structure, i.e., the R and S configurations for each asymmetric center. Therefore, unless otherwise indicated, single stereochemical isomers as well as enantiomeric, racemic and diastereomeric mixtures of the present chemical entities are within the scope of the invention. When a stereochemical configuration is denoted for a compound, the diastereoisomeric or enantiomeric excess of the compound is in some embodiments, at least 99.0%, 99.5%, 99.6%, 99.7%, 99.8% or 99.9%.

Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids).

The present disclosure provides a combination treatment for patients with cancer. The combination treatment comprises administering to a subject in need thereof a therapeutically effective amount of at least one EZH2 modulator, for example an EZH2 inhibitor.

EZH2 modulators described herein include e.g., small molecules that are capable of modulating EZH2 activity such as e.g., small molecule inhibitors of EZH2. Modulation can be measured in vitro, in vivo, or from a combination thereof. In one aspect, the EZH2 modulators described herein include, but are not limited to,

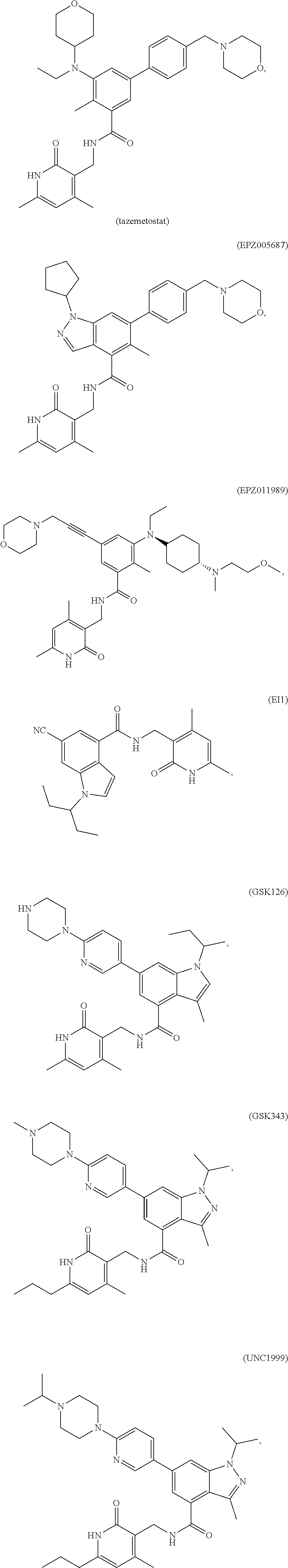

as well as those described in WO 2022/0135582, WO 2022/035303, WO 2020/171606, WO 2019/094552, WO 2019/204490, WO 2019/226491, and WO 2021/016414, each of which are incorporated herein by reference.

In some embodiments, the EZH2 modulator is of the Formula I:

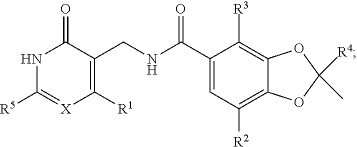

or a pharmaceutically acceptable salt thereof, wherein:
   R¹ is halo, —S(C₁-C₄)alkyl, —S(C₃-C₇)cycloalkyl, or —S[halo(C₁-C₄)alkyl];
   X is CH or N;
   R² is hydrogen, halo, (C₁-C₄)alkyl, or halo(C₁-C₄)alkyl;
   R³ is halo, (C₁-C₄)alkyl, or halo(C₁-C₄)alkyl;
   R⁴ is (C₃-C₇)cycloalkyl or 4-7 membered heterocyclyl, each of which are optionally substituted with 1 to 3 groups selected from halo, (C₁-C₄)alkyl, halo(C₁-C₄)alkyl, (C₁-C₄)alkoxy, halo(C₁-C₄)alkoxy, and —NR^{a}R^{b};
R^{a} is hydrogen, (C₁-C₄)alkyl, or halo(C₁-C₄)alkyl;
   R^{b} is (C₁-C₄)alkyl, halo(C₁-C₄)alkyl, or 4-7 membered heterocyclyl, wherein said heterocyclyl is optionally substituted with 1 to 3 groups selected from halo, (C₁-C₄)alkyl, halo(C₁-C₄)alkyl, (C₁-C₄)alkoxy, and halo(C₁-C₄)alkoxy; or
   R^{a} and R^{b} together with the nitrogen atom to which they are attached form a 4-7 membered heterocyclyl optionally substituted with 1 to 3 groups selected from halo, (C₁-C₄)alkyl, halo(C₁-C₄)alkyl, and —OR^{c};
   R^{c} is (C₁-C₄)alkyl, halo(C₁-C₄)alkyl, or (C₃-C₇)cycloalkyl; and
   R⁵ is halo, (C₁-C₄)alkyl, or halo(C₁-C₄)alkyl.

In some embodiments, the EZH2 modulator is of the Formula I, or a pharmaceutically acceptable salt thereof, wherein
R¹ is halo or —S(C₁-C₄)alkyl;
R² is halo; R³ is (C₁-C₄)alkyl;
R⁴ is (C₃-C₇)cycloalkyl or (C₄-C₇) heterocyclyl, each of which are optionally substituted with 1 to 2 groups selected from halo(C₁-C₄)alkyl and —NR^{a}R^{b}; R^{a} is hydrogen or (C₁-C₄)alkyl;
R^{b} is (C₁-C₄)alkyl or (C₄-C₇) heterocyclyl, wherein said heterocyclyl is optionally substituted with halo(C₁-C₄)alkyl; or
R^{a} and R^{b} together with the nitrogen atom to which they are attached form a 4-7 membered nitrogen containing heterocyclyl optionally substituted with 1 to 3 groups selected from halo and —OR^{c};
R^{c} is (C₁-C₄)alkyl, halo(C₁-C₄)alkyl, or (C₃-C₇)cycloalkyl; and
R⁵ is halo or (C₁-C₄)alkyl.

In some embodiments, the EZH2 modulator is of the Formula II:

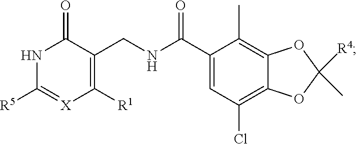

or a pharmaceutically acceptable salt thereof, wherein the variables are as described in any of the above embodiments.

In some embodiments, R¹ in the EZH2 modulator of Formula I or II, or a pharmaceutically acceptable salt thereof, is chloro, wherein the remaining variables are as described in any of the above embodiments.

In some embodiments, R¹ in the EZH2 modulator of Formula I or II, or a pharmaceutically acceptable salt thereof, is-SCH3, wherein the remaining variables are as described in any of the above embodiments.

In some embodiments, R⁴ in the EZH2 modulator of Formula I or II, or a pharmaceutically acceptable salt thereof, is cyclohexyl or piperidinyl, each of which are optionally substituted with 1 to 2 groups selected from halo(C₁-C₄)alkyl and —R^{a}R^{b}, wherein the remaining variables are as described in any of the above embodiments.

In some embodiments, the EZH2 modulator is of the Formula III:

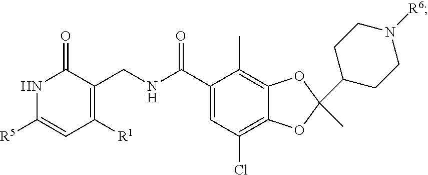

or a pharmaceutically acceptable salt thereof, wherein R⁶ is halo(C₁-C₄)alkyl and wherein the remaining variables are as described in any of the above embodiments.

In some embodiments, the EZH2 modulator is of the Formula IV:

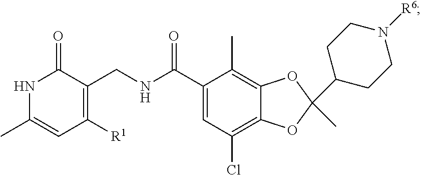

or a pharmaceutically acceptable salt thereof, wherein the variables are as described in any of the above embodiments.

In some embodiments, the EZH2 modulator is of the Formula V:

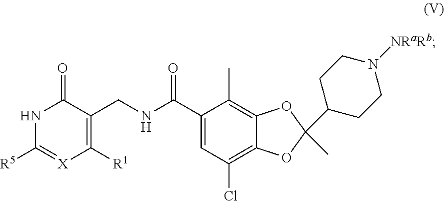

or a pharmaceutically acceptable salt thereof, wherein the variables are as described in any of the above embodiments.

In some embodiments, the EZH2 modulator is of the Formula VI or VII:

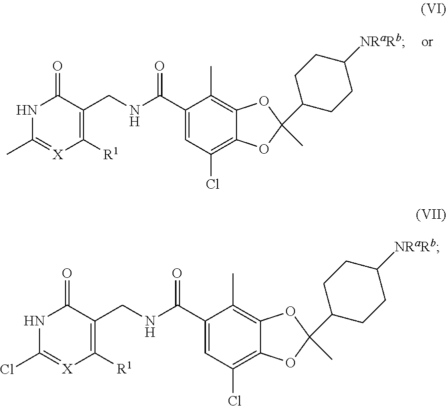

or a pharmaceutically acceptable salt thereof, wherein the variables are as described in any of the above embodiments.

In some embodiments, R^{b} in the above described EZH2 modulators and pharmaceutically acceptable salts thereof, is (C₁-C₄)alkyl or oxetanyl, wherein said oxetanyl is optionally substituted with halo(C₁-C₄)alkyl; or R^{a} and R^{b} together with the nitrogen atom to which they are attached form an azetidinyl optionally substituted with halo or —OR^{c} and wherein the remaining variables are as described in any of the above embodiments.

In some embodiments, R^{a} in the above described EZH2 modulators and pharmaceutically acceptable salts thereof, is hydrogen or methyl; and R^{b} is methyl or oxatanyl, wherein said oxetanyl is optionally substituted with —CH₂F or —CF₃ and wherein the remaining variables are as described in any of the above embodiments.

In some embodiments, R^{a} and R^{b} in the above described EZH2 modulators and pharmaceutically acceptable salts thereof, together with the nitrogen atom to which they are attached form an azetidinyl optionally substituted with 1 to 2 fluoro or —OR^{c}; and R^{c} is —CH₃, —CHF₂, or cyclopropyl, wherein the remaining variables are as described in any of the above embodiments.

In some embodiments, the EZH2 modulator is of the following chemical structure:

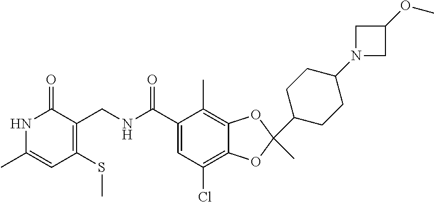

or a pharmaceutically acceptable salt thereof.

In some embodiments, the EZH2 modulator is of the following chemical structure:

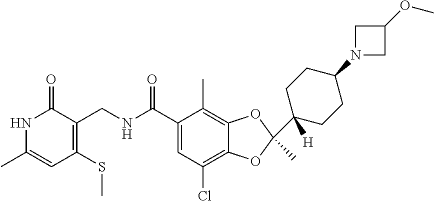

(also referred to herein as Compound 1) or a pharmaceutically acceptable salt thereof.

In some embodiments, the EZH2 modulator is crystalline Form 1 of the following chemical structure:

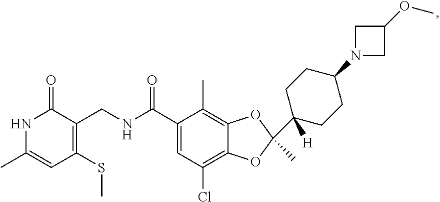

wherein the crystalline form is characterized by at least three x-ray powder diffraction peaks at 2θ angles selected from 10.0°, 13.3°, 14.9°, 20.2°, 20.8°, 22.2°, and 22.5°; by at least four x-ray powder diffraction peaks at 2θ angles selected from 10.0°, 13.3°, 14.9°, 20.2°, 20.8°, 22.2°, and 22.5°; by at least five x-ray powder diffraction peaks at 2θ angles selected from 10.0°, 13.3°, 14.9°, 20.2°, 20.8°, 22.2°, and 22.5°; by at least six x-ray powder diffraction peaks at 2θ angles selected from 10.0°, 13.3°, 14.9°, 20.2°, 20.8°, 22.2°, and 22.5°; by x-ray powder diffraction peaks at 2θ angles selected from 10.0°, 13.3°, 14.9°, 20.2°, 20.8°, 22.2°, and 22.5°; by x-ray powder diffraction peaks at 2θ angles selected from 10.0°, 10.2°, 12.3°, 12.7°, 13.3°, 14.9°, 15.3°, 20.2°, 20.8°, 21.3°, 22.2°, 22.5°, and 23.8°; or by x-ray powder diffraction peaks at 2θ angles selected from 10.0°, 10.2°, 11.0°, 11.4°, 11.8°, 12.3°, 12.7°, 13.3°, 14.9°, 15.3°, 16.1°, 17.4°, 20.2°, 20.8°, 21.3°, 22.2°, 22.5°, and 23.8°.

In some embodiments, the EZH2 modulator is crystalline Form 1 of the following chemical structure:

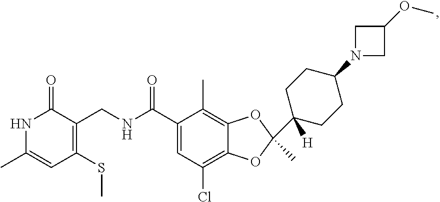

wherein the crystalline form is characterized by x-ray powder diffraction peaks at 2θ angles selected from 14.9°, 20.2°, and 20.8°; by x-ray powder diffraction peaks at 2θ angles selected from 10.0°, 14.9°, 20.2°, and 20.8°; by x-ray powder diffraction peaks at 2θ angles selected from 10.0°, 14.9°, 20.2°, 20.8°, and 22.2°; or by x-ray powder diffraction peaks at 2θ angles selected from 10.0°, 13.3°, 14.9°, 20.2°, 20.8°, and 22.2°.

The present disclosure provides a combination treatment that includes, inter alia, administering to a subject in need thereof a therapeutically effective amount of at least one anti-CD19 antibody (e.g., tafasitamab).

The use of anti-CD19 antibodies in non-specific B cell lymphomas is discussed in WO2007076950 (US2007154473), which are both incorporated by reference in their entirety. The use of a CD19-antibody in CLL, NHL and ALL is described in Scheuermann et al., CD19 Antigen in Leukemia and Lymphoma Diagnosis and Immunotherapy, Leukemia and Lymphoma, Vol. 18, 385-397 (1995), which is incorporated by reference in its entirety.

Additional antibodies specific for CD19 are described in WO2005012493 (U.S. Pat. No. 7,109,304), WO2010053716 (U.S. Ser. No. 12/266,999) (Immunomedics); WO2007002223 (US U.S. Pat. No. 8,097,703) (Medarex); WO2008022152 (U.S. Ser. No. 12/377,251) and WO2008150494 (Xencor), WO2008031056 (U.S. Ser. No. 11/852,106) (Medimmune); WO 2007076950 (U.S. Ser. No. 11/648,505) (Merck Patent GmbH); WO 2009/052431 (U.S. Ser. No. 12/253,895) (Seattle Genetics); and WO2010095031 (Ser. No. 12/710,442) (Glenmark Pharmaceuticals), WO2012010562 and WO2012010561 (International Drug Development), WO2011147834 (Roche Glycart), and WO2012156455 (Sanofi), which are all incorporated by reference in their entireties.

The dose of an antibody comprised in a pharmaceutical composition comprising a combination according to the present disclosure administered to a patient may vary depending upon the age and the size of the patient, symptoms, conditions, route of administration, and the like. The dose is typically calculated according to body weight, or body surface area, age, or per individual. Depending on the severity of the condition, the frequency and the duration of the treatment can be adjusted. Effective dosages and schedules for administering pharmaceutical compositions comprising antibodies or antibody fragments specific for CD19 may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

The anti-CD19 antibody can be administered as a fixed dose, or in a mg/kg patient weight dose. The dose can also be chosen to reduce or avoid production of antibodies against the anti-CD19 antibody. Dosage regimens are adjusted to provide the desired response, e.g., a therapeutic response or a combinatorial therapeutic effect. Generally, doses of the anti-CD19 antibody can be used in order to provide a subject with the agent in bioavailable quantities.

The anti-CD19 antibody can be administered once a week, once every two weeks or once every four weeks.

For example, doses in the range of about 9 mg/kg to about 30 mg/kg can be administered. In specific embodiments, a subject in need of treatment with an anti-CD19 antibody is administered the antibody at a dose of about 9 mg/kg or about 30 mg/kg. With respect to doses or dosages, the term “about” is intended to denote a range that is +10% of a recited dose, such that, for example, a dose of about 3 mg/kg will be between 2.7 mg/kg and 3.3 mg/kg patient weight.

The anti-CD19 antibody can be administered at a dose of at least 9 mg/kg. In another embodiment the anti-CD19 antibody can be administered at a dose of 9 mg/kg to 12 mg/kg. In another embodiment the anti-CD19 antibody can be administered at a dose of 9 mg/kg.

The anti-CD19 antibody can be administered at a dose of 24 mg/kg or more. In another embodiment the anti-CD19 antibody can be administered at a dose of 24 mg/kg to 30 mg/kg. In another embodiment the anti-CD19 antibody can be administered at a dose of 24 mg/kg. In another embodiment the anti-CD19 antibody can be administered at a dose of 30 mg/kg. The pharmaceutical composition may include dosage forms for intravenous, subcutaneous, intracutaneous and intramuscular injections. These injectable preparations may be prepared by known methods. For example, the injectable preparations may be prepared, e.g., by dissolving, suspending or emulsifying the antibody described above or its salt in a sterile aqueous medium or an oily medium conventionally used for injections. Exemplary pharmaceutical compositions comprising an antibody specific for CD19 that can be used in the context of the present disclosure are disclosed, e.g., in WO2008/022152 or WO2018/002031 which are both incorporated by reference in their entireties.

In certain ways of administration used with embodiments of the present disclosure, e.g. intravenous administration, it is preferred to administer drug depending on the body weight of the patient. In other ways of administration used with embodiments of the present disclosure, e.g. subcutaneous administration, it is preferred to administer drug at a flat, fixed does. The skilled person is aware of which dose in one way of administration is equivalent or substantially equivalent to another dose in another way of administration. For example, the pharmacodynamics of a specific drug are typically taken into account in a reasoned decision to administer a drug in the required form and at a required, efficacious dose. The antibody which is administered according to the present disclosure can be administered to the patient in a therapeutically effective amount.

In some embodiments, the present disclosure relates to a method of treating a cancer in a patient by administering to a patient in need of said treatment a combination of an EZH2 modulator and one or more anti-CD19 antibodies.

In some embodiments, the present disclosure relates to a method of treating a cancer by administering to a patient in need of said treatment a combination of an EZH2 modulator and an anti-CD19 antibody.

In some embodiments, the present disclosure relates to the use of an EZH2 modulator in combination with an anti-CD19 antibody for the treatment of a cancer in a patient.

In some embodiments, the present disclosure relates to a composition comprising an EZH2 modulator for use in treating a cancer in a patient, wherein the patient is also treated with an anti-CD19 antibody. In some aspects, the disclosure relates to a composition comprising an EZH2 modulator for use in treating a disorder which is cancer in a patient, wherein the EZH2 modulator is in combination with the anti-CD19 antibody. In some embodiments, the EZH2 modulator can be administered simultaneously or sequentially with the anti-CD19 antibody.

In some embodiments, the present disclosure relates to methods of treating a cancer comprising administering to a patient in need of such treatment, a therapeutically effective amount of a combination of an EZH2 modulator and an anti-CD19 antibody.

In some embodiments, the present disclosure relates to a method of treating a cancer by administering to a patient a combination of an EZH2 modulator and an anti-CD19 antibody.

In another aspect, the present disclosure relates to the use of an EZH2 modulator in combination with an anti-CD19 antibody for the treatment of a cancer.

In some embodiments, the methods of treating a cancer, as described herein, can include a combination of an EZH2 modulator, an anti-CD19 antibody, and one or more additional therapeutic agents. In some embodiments, the one or more additional therapeutic agents can be chemotherapeutic agents. In some embodiments, the one or more additional therapeutic agents can include, but are not limited to lenalidomide, fludarabine, cyclophosphamide, doxorubicin, vincristine, methotrexate anthracycline-based chemotherapeutic agents, prednisone, methylprednisolone, glucocorticoids, Ibritumomab tiuxetan, acetaminophen, antihistamines, and combinations thereof. In some embodiments, the one or more additional therapeutic agents can include R—CHOP.

In an embodiment, the one or more additional therapeutic agent is lenalidomide.

In some embodiments, the cancer is a CD19-positive cancer.

In some embodiments, the cancer is a hematological cancer. Non-limiting examples of hematologic cancers include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin's lymphoma (HL); non-Hodgkin's lymphoma (NHL), including B-cell lymphoma, T-cell lymphoma, follicular lymphoma (FL), marginal zone lymphoma (MZL), mantle cell lymphoma (MCL), diffuse large B-cell lymphoma (DLBCL), and Burkitt lymphoma; multiple myeloma (MM); amyloidosis; Waldenstrom's macroglobulinemia; myelodysplastic syndromes (MDS), including refractory anemia (RA), refractory anemia with ringed sideroblasts (RARS), (refractory anemia with excess blasts (RAEB), and RAEB in transformation (RAEB-T); and myeloproliferative syndromes. In some embodiments, the cancer is chronic lymphocytic leukemia (CLL), Hodgkin's lymphoma, or non-Hodgkin's lymphoma including follicular lymphoma (FL), marginal zone lymphoma (MZL), mantle cell lymphoma (MCL), diffuse large B-cell lymphoma (DLBCL) and Burkitt lymphoma.

In some embodiments, the cancer is chronic lymphocytic leukemia. In some embodiments, the cancer is CD19-positive chronic lymphocytic leukemia.

In some embodiments, the cancer is a non-Hodgkin's lymphoma, including follicular lymphoma (FL), marginal zone lymphoma (MZL), mantle cell lymphoma (MCL), Diffuse large B-cell lymphoma (DLBCL), and Burkitt lymphoma. In some embodiments, the cancer is a B-cell lymphoma.

In one embodiment, the lymphoma is Non-Hodgkin's lymphoma. In another embodiment, the Non-Hodgkin's lymphoma is DLBCL. In another embodiment, the DLBCL is relapsed, refractory, or relapsed and refractory DLBCL. In one embodiment, the DLBCL is relapsed DLBCL. In one embodiment, the DLBCL is refractory DLBCL. In one embodiment, the DLBCL is relapsed and refractory DLBCL. In another embodiment, the DLBCL is relapsed, refractory DLBCL wherein the patients suffering relapsed, refractory DLBCL are not eligible for autologous stem cell transplantation (ASCT).

In another embodiment, the DLBCL is primary refractory DLBCL.

In one embodiment, the DLBCL is first line DLBCL. In another embodiment, the DLBCL is newly diagnosed DLBCL. In another embodiment, the DLBCL is newly diagnosed DLBCL wherein the patients suffering newly diagnosed DLBCL are at least 80 years old and are not eligible for autologous stem cell transplantation (ASCT). In another embodiment, the DLBCL is newly diagnosed frail/unfit DLBCL. In another embodiment the DLBCL is newly diagnosed DLBCL wherein the patients suffering newly diagnosed DLBCL are frail with an age above or equal 80 years. In another embodiment the DLBCL is newly diagnosed DLBCL wherein the patients suffering newly diagnosed DLBCL are frail with an age above or equal 80 years, an ADL score of 6 and an IADL score of 8. In another embodiment the DLBCL is newly diagnosed DLBCL wherein the patients suffering newly diagnosed DLBCL are unfit with an age above or equal 65 years and with at least one of the following comorbidities: (i) (LVEF) ≥30 to <50%, (ii) history of myocardial infarction within 6 months prior to screening, (iii) ischemic heart disease, (iv) history of stroke within 12 months prior to screening.

In another embodiment the DLBCL is newly diagnosed DLBCL wherein the patients suffering newly diagnosed DLBCL are (a) frail with an age above or equal 80 years, an ADL score of 6 and an IADL score of 8 or (b) are unfit with an age above or equal 65 years with at least one of the following comorbidities: (i) (LVEF) ≥30 to <50%, (ii) History of myocardial infarction within 6 months prior to screening, (iii) ischemic heart disease, (iv) history of stroke within 12 months prior to screening.

In another embodiment, the DLBCL is newly diagnosed DLBCL. In another embodiment the DLBCL is newly diagnosed DLBCL wherein the patients suffering newly diagnosed DLBCL are at least 80 years old. In another embodiment the DLBCL is newly diagnosed DLBCL wherein the patients suffering newly diagnosed are at least 80 years old, have an ADL score of 6 and an IADL score of 8. In another embodiment the DLBCL is newly diagnosed DLBCL wherein the patients suffering newly diagnosed DLBCL are at least 65 years old and have at least one of the following comorbidities: (i) (LVEF) ≥30 to <50%, (ii) history of myocardial infarction within 6 months prior to screening, (iii) ischemic heart disease, (iv) history of stroke within 12 months prior to screening.

In another embodiment the DLBCL is newly diagnosed DLBCL wherein the patients suffering newly diagnosed DLBCL are (a) at least 80 years old, have an ADL score of 6 and an IADL score of 8 or (b) are at least 65 years old and have at least one of the following comorbidities: (i) (LVEF) ≥30 to <50%, (ii) history of myocardial infarction within 6 months prior to screening, (iii) ischemic heart disease, (iv) history of stroke within 12 months prior to screening.

In one embodiment, the lymphoma is a CD20-expressing lymphoma. In another embodiment, the lymphoma is a CD19-expressing lymphoma. In another embodiment, the lymphoma is a CD20-expressing and CD19-expressing lymphoma.

In one embodiment, the subject has received a prior CAR-T therapy.

In some embodiments, the cancer is a CD19-positive non-Hodgkin's lymphoma.

In some embodiments, the cancer is a CD19-positive aggressive non-Hodgkin's lymphoma. In some embodiments, the cancer is a CD19-positive indolent non-Hodgkin's lymphoma. In some embodiments, the cancer is relapsed or refractory non-Hodgkin's lymphoma. In some embodiments, the cancer is relapsed or refractory CD19-positive aggressive non-Hodgkin's lymphoma. In some embodiments, the cancer is relapsed or refractory CD19-positive indolent non-Hodgkin's lymphoma.

In some embodiments, the cancer is relapsed. In some embodiments, relapsed cancer is cancer which has returned after a period of time in which no cancer could be detected.

In some embodiments, the cancer is refractory. In some embodiments, refractory cancer does not respond to cancer treatment; it is also known as resistant cancer. In some embodiments, the cancer is resistant to rituximab. In some embodiments, the cancer does not respond to the treatment of rituximab. In some embodiments, the cancer is rituximab-resistant recurrent cancer. In some embodiments, the patient has become refractory to a rituximab-containing regimen. In some embodiments, the tumor is unresectable. In some embodiments, the cancer has not been previously treated. In some embodiments, the cancer is locally advanced. In some embodiments, “locally advanced” refers to cancer that is somewhat extensive but still confined to one area. In some instances, “locally advanced” may refer to a small tumor that hasn't spread but has invaded nearby organs or tissues that make it difficult to remove with surgery alone. In some embodiments, the cancer is metastatic.

In some embodiments, the patient has relapsed or refractory CD19-positive non-Hodgkin lymphoma. In some embodiments, the patient has both CD19-positive non-Hodgkin's lymphoma and relapsed or refractory non-Hodgkin's lymphoma.

In some embodiments, the patient has relapsed or refractory CD19-positive aggressive non-Hodgkin's lymphoma. In some embodiments, the patient has relapsed or refractory CD19-positive aggressive non-Hodgkin's lymphoma and has progressed on at least one prior treatment regimen.

In some embodiments, the patient has relapsed or refractory CD19-positive indolent non-Hodgkin's lymphoma. In some embodiments, the patient has relapsed or refractory CD19-positive indolent non-Hodgkin's lymphoma and has progressed on at least two prior treatment regimens. In some embodiments, the patient has relapsed or refractory CD19-positive indolent non-Hodgkin's lymphoma and is refractory to any anti-CD20 monoclonal antibody or any anti-CD19 monoclonal antibody. In some embodiments, the patient has relapsed or refractory CD19-positive indolent non-Hodgkin's lymphoma and has progressed on at least two prior treatment regimens and is refractory to an anti-CD20 monoclonal antibody.

In some embodiments, the present disclosure relates to a medicament for use in treating a cancer in a patient in need of such treatment. The medicament comprises an EZH2 modulator and an anti-CD19 antibody, and is in single dosage form or in separate dosage forms.

In some embodiments, the medicaments, as described herein, can include a combination of an EZH2 modulator, an anti-CD19 antibody, and optionally one or more additional therapeutic agents.

In some embodiments, the present disclosure relates to the use of an EZH2 modulator in the manufacture of a medicament for treating a cancer, wherein the EZH2 modulator is administered with an anti-CD19 antibody, and wherein the medicament is in single dosage form or in separate dosage forms. In some embodiments, the EZH2 modulator is administered with an anti-CD19 antibody and one or more additional therapeutic agents.

In some embodiments, the present disclosure relates to the use of an EZH2 modulator for the manufacture of a medicament in treating a cancer in a patient, wherein the patient is also treated with an anti-CD19 antibody, and optionally one or more additional therapeutic agents. In some embodiments, the EZH2 modulator may be administered simultaneously or sequentially with the anti-CD19 antibody. In some embodiments, the EZH2 modulator is in the same composition as the anti-CD19 antibody. In some embodiments, the EZH2 modulator is in a separate composition as the anti-CD19 antibody. In some embodiments, the EZH2 modulator is in the same composition as one or more additional therapeutic agents. In some embodiments, the EZH2 modulator is in the same composition as the anti-CD19 antibody, and optionally one or more additional therapeutic agents. In some embodiments, the EZH2 modulator is in a separate composition as one or more additional therapeutic agents. In some embodiments, the EZH2 modulator is in a separate composition as the anti-CD19 antibody, and optionally one or more additional therapeutic agents.

In another aspect, the present disclosure relates to the use of an EZH2 modulator in combination with an anti-CD19 antibody in the manufacture of a medicament for use in treating a cancer. In some embodiments, the present disclosure relates to the use of an EZH2 modulator in combination with an anti-CD19 antibody, and optionally one or more additional therapeutic agents in the manufacture of a medicament for use in treating a cancer.

In another aspect, the present disclosure relates to the use of an EZH2 modulator in the manufacture of a medicament for treating a cancer, wherein the EZH2 modulator is administered with an anti-CD19 antibody, and optionally one or more additional therapeutic agents.

In one aspect, the present disclosure relates to an anti-CD19 antibody for use in the treatment of a cancer, wherein said anti-CD19 antibody is administered in combination with an EZH2 modulator to a subject in need of such treatment.

In one aspect, the present disclosure relates to an anti-CD19 antibody for use in the treatment of a cancer, wherein said anti-CD19 antibody is administered in combination with an EZH2 modulator.

In some embodiments, the anti-CD19 antibody comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising the sequence LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

In some embodiments, the anti-CD19 antibody comprises a heavy chain variable region comprising an HCDR1 region of SYVMH (SEQ ID NO: 1), an HCDR2 region of NPYNDG (SEQ ID NO: 2), and an HCDR3 region of GTYYYGTRVFDY (SEQ ID NO: 3) and a light chain variable region comprising an LCDR1 region of RSSKSLQNVNGNTYLY (SEQ ID NO: 4), an LCDR2 region of RMSNLNS (SEQ ID NO: 5), and an LCDR3 region of MQHLEYPIT (SEQ ID NO: 6).

In some embodiments, the anti-CD19 antibody comprises a heavy chain variable region of

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS
and
a light chain variable region of
(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

In some embodiments, the anti-CD19 antibody has effector function. In another aspect the antibody or antibody fragment specific for CD19 has an enhanced effector function. In one embodiment the effector function is ADCC. In one embodiment the antibody or antibody fragment specific for CD19 has an enhanced ADCC activity. In a further embodiment the antibody or antibody fragment specific for CD19 comprises an Fc domain comprising an amino acid substitution at position S239 and/or 1332, wherein the numbering is according to the EU index as in Kabat.

In some embodiments, the anti-CD19 antibody comprises a heavy chain constant region of

(SEQ ID NO: 9)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPDVFLFPPKPKDTLMISRTPEVTCVVVDVS
HEDPEVQFNWYVDGVEVHNAKTKPREEQFNSTFRVVSVLTVVHQDWLNGK
EYKCKVSNKALPAPEEKTISKTKGQPREPQVYTLPPSREEMTKNQVSLTC
LVKGFYPSDIAVEWESNGQPENNYKTTPPMLDSDGSFFLYSKLTVDKSRW
QQGNVFSCSVMHEALHNHYTQKSLSLSPGK.

In some embodiments, the anti-CD19 antibody comprises a light chain constant region of

(SEQ ID NO: 10)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In some embodiments, the anti-CD19 antibody comprises a heavy chain constant region of

(SEQ ID NO: 9)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPDVFLFPPKPKDTLMISRTPEVTCVVVDVS
HEDPEVQFNWYVDGVEVHNAKTKPREEQFNSTFRVVSVLTVVHQDWLNGK
EYKCKVSNKALPAPEEKTISKTKGQPREPQVYTLPPSREEMTKNQVSLTC
LVKGFYPSDIAVEWESNGQPENNYKTTPPMLDSDGSFFLYSKLTVDKSRW
QQGNVFSCSVMHEALHNHYTQKSLSLSPGK
and
a light chain constant region of
(SEQ ID NO: 10)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In some embodiments, the anti-CD19 antibody comprises a heavy chain region of

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K
and
a light chain region of
(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

In some embodiments, the anti-CD19 antibody is tafasitamab.

An EZH2 modulator, may be administered in combination with the anti-CD19, and optionally one or more additional therapeutic agents, in a single dosage form or as a separate dosage forms. In some embodiments, when administered as a separate dosage form, the anti-CD19 antibody may be administered prior to, at the same time as, or following administration of an EZH2 modulator. In some embodiments, when administered as a separate dosage form, one or more doses of an EZH2 modulator may be administered prior to the anti-CD19 antibody. In some embodiments, the anti-CD19 antibody is administered prior to the administration of an EZH2 modulator. As used herein, the administration in “combination” of an EZH2 modulator, an anti-CD19 antibody, and optionally one or more additional therapeutic agents refers not only to simultaneous or sequential administration of the agents, but also to the administration of the agents during a single treatment cycle, as understood by one skilled in the art. When an EZH2 modulator is administered in combination with the anti-CD19 antibody, and optionally one or more additional therapeutic agents, a therapeutically effective amount of the combination can be administered.

The EZH2 modulator may be administered by any method known to one skilled in the art. For example, in some embodiments, the EZH2 modulator may be administered in the form of a pharmaceutical composition of the EZH2 modulator and a pharmaceutically acceptable carrier, such as those described herein. In some embodiments, the pharmaceutical composition is suitable for oral administration. In some embodiments, the pharmaceutical composition is a tablet or a capsule that is suitable for oral administration. In some other embodiments, the pharmaceutical composition is a liquid dosage form suitable for oral administration. In some embodiments, the pharmaceutical composition is suitable for intravenous administration. In some embodiments, the pharmaceutical composition is suitable for subcutaneous administration. In some embodiments, these compositions optionally further comprise one or more additional therapeutic agents.

The anti-CD19 antibody may be administered by any method known to one skilled in the art. In some embodiments, the anti-CD19 antibody is administered intravenously (IV). In some embodiments, the anti-CD19 antibody is administered subcutaneously (SC). In some embodiments, the anti-CD19 antibody is administered orally. For example, the anti-CD19 antibody may be administered in the form of a second composition, in some embodiments, a pharmaceutical composition of the anti-CD19 antibody and a pharmaceutically acceptable carrier, such as those described herein. In some aspects, the pharmaceutical composition is suitable for oral administration. In some embodiments, the pharmaceutical composition is a tablet or a capsule that is suitable for oral administration. In some other embodiments, the pharmaceutical composition is a liquid dosage form suitable for oral administration. In some embodiments, these compositions optionally further comprise one or more additional therapeutic agents.

The amounts or suitable doses of the methods of this disclosure depends upon a number of factors, including the nature of the severity of the condition to be treated, the particular inhibitor, the route of administration and the age, weight, general health, and response of the individual patient. In some embodiments, the suitable dose level is one that achieves a therapeutic response as measured by tumor regression, or other standard measures of disease progression, progression free survival or overall survival. In some embodiments, the suitable dose level is one that achieves this therapeutic response and also minimizes any side effects associated with the administration of the therapeutic agent. The suitable dose levels may be ones that prolong the therapeutic response and/or prolong life.

It will be understood that a suitable dose of the EZH2 modulator, such as Compound I, that will elicit a biological or medical response of a subject e.g., a dosage of between 0.01-100 mg/kg body weight/day can be administered. In one embodiment, the dose of the EZH2 modulator, such as Compound I, ranges from about 10 mg/kg body weight/day to about 150 mg/kg body weight/day. In another embodiment, the dose of the EZH2 modulator, such as Compound I, ranges from about 50 mg to about 375 mg daily. In another embodiment, the dose of the EZH2 modulator, such as Compound I, is about 375 mg daily. In another embodiment, the dose of the EZH2 modulator, such as Compound I, is 375 mg daily.

It will be understood that a suitable dose of the EZH2 modulator, the anti-CD19 antibody, and optionally one or more additional therapeutic agents may be taken at any time of the day or night. In some embodiments, a suitable dose of each agent is taken in the morning. In some other embodiments, a suitable dose of each agent is taken in the evening. In some embodiments, a suitable dose of each of the agents is taken both in the morning and the evening. It will be understood that a suitable dose of each agent may be taken with or without food. In some embodiments a suitable dose of an agent is taken with a meal. In some embodiments a suitable dose of an agent is taken while fasting.

In some embodiments, an EZH2 modulator is administered on a daily schedule. In some embodiments, an EZH2 modulator is administered every other day. In some embodiments, an EZH2 modulator is administered once every three days. In some embodiments, an EZH2 modulator is administered on a twice-weekly schedule. In some embodiments, an EZH2 modulator is administered on a three times a week schedule. In some embodiments, an EZH2 modulator is administered on a weekly schedule. In some embodiments, an EZH2 modulator is administered on a once every two weeks schedule.

In some embodiments, an EZH2 modulator is administered at least 3 times on alternate days within a 7-day cycle. In some embodiments, an EZH2 modulator is administered on day 1 and day 4 of a 7-day cycle. In some embodiments, an EZH2 modulator is administered on consecutive days in a 7-day cycle followed by an intermission. In some embodiments, an EZH2 modulator is administered for 2 consecutive days followed by an intermission of 5 consecutive days for at least one 7-day cycle. In some embodiments, an EZH2 modulator is administered for 3 consecutive days followed by an intermission of 4 consecutive days for at least one 7-day cycle. In some embodiments, an EZH2 modulator is administered for 4 consecutive days followed by an intermission of 3 consecutive days for at least one 7-day cycle. In some embodiments, an EZH2 modulator is administered for 5 consecutive days followed by an intermission of 2 consecutive days for at least one 7-day cycle. In some embodiments, there will be periods of rest between one or more of the 7-day treatment cycles. In some embodiments, there will be a 7-day rest between one or more of the 7-day treatment cycles.

The present description contemplates administration of the drug for one or more treatment cycles, for example, 1, 2, 3, 4, 5, 6, or more, treatment cycles. In some embodiments, a treatment cycle is about 7 days to about 56 days, or more. In some embodiments, a treatment cycle is 7 days, 14 days, 21 days, 28 days, 35 days, 42 days, 49 days, or 56 days. In some embodiments, a treatment cycle is 21 days or 28 days. In some embodiments, there will be periods of rest within or between one or more of the treatment cycles. For example, in some embodiments, there will be a period of rest at the end of the treatment cycle. In some embodiments, there will be a period of rest between the second and third treatment cycle but not the first and second treatment cycle. In another embodiment, there might be a period of rest between the first and second treatment cycle but not the second and third treatment cycle. Dosing schedules include, for example, administering the EZH2 modulator once during a treatment schedule, e.g., on day 1 of a 21 day cycle, twice during a treatment cycle, e.g., on days 1 and 15 of a 21 day cycle or on days 1 and 15 of a 28 day cycle, three times during a treatment cycle, e.g., on days 1, 8 and 15 of a 21 day cycle or on days 1, 8 and 15 of a 28 day cycle, and four times during a treatment cycle, e.g., on days 1, 4, 8, and 11 of a 21 day cycle or on days 1, 4, 8, and 11 of a 28 day cycle. Other dosage schedules are encompassed by the present invention.

In some embodiments, an EZH2 modulator is administered within a 21-day cycle. In some embodiments, an EZH2 modulator is administered at least two times within a 21-day cycle. In some embodiments, an EZH2 modulator is administered on day 1 within a 21-day cycle. In some embodiments, an EZH2 modulator is administered on day 8 within a 21-day cycle. In some embodiments, an EZH2 modulator is administered on days 1 and 8 within a 21-day cycle.

In some embodiments, an EZH2 modulator is administered for a duration of 1 year or less. In some embodiments, an EZH2 modulator is administered for a duration of 1 year or more.

In some embodiments, the EZH2 modulator or the anti-CD19 antibody described herein may be manufactured for inclusion in a kit. A “kit” is any article of manufacture (e.g., a package or container) comprising at least one reagent or chemotherapeutic agent. A kit for use in the methods herein may comprise an EZH2 modulator, such as a compound of formula I or a pharmaceutically acceptable salt thereof. In some embodiments, the kit may further include an anti-CD19 antibody, and optionally one or more additional therapeutic agents. In some embodiments, the kit may include a compound of formula I or a pharmaceutically acceptable salt thereof, an anti-CD19 antibody, and optionally one or more additional therapeutic agents. In some embodiments, the kit may include one or more EZH2 modulators or pharmaceutically acceptable salts thereof. In some embodiments, the kit may include one or more anti-CD19 antibodies.

In some embodiments, the present disclosure relates to a kit comprising a medicament for use in treating cancer in a patient in need of such treatment. The kit can comprise a medicament comprising an EZH2 modulator, and optionally instructions for administering the EZH2 modulator and an anti-CD19 antibody; or the kit can comprise a medicament comprising an anti-CD19 antibody, and optionally instructions for administering the anti-CD19 antibody and an EZH2 modulator. The kit may contain a medicament comprising an EZH2 modulator and an anti-CD19 antibody, and optionally instructions for administering the EZH2 modulator and the anti-CD19 antibody, wherein the medicament is in single dosage form or in separate dosage forms. In some embodiments, the kit optionally comprises one or more additional therapeutic agents.

In some embodiments, a kit comprising an EZH2 modulator and an anti-CD19 antibody may further include another component or reagent. In some embodiments, a reagent in the kit may be a diluent for preparing the EZH2 modulator for administration. In some embodiments, a reagent in the kit may be a diluent for preparing the anti-CD19 antibody for administration. In some embodiments, a component in the kit may be a vessel for mixing the combination of the EZH2 modulator and the anti-CD19 antibody.

In another aspect, the present disclosure relates to a kit for treating cancer comprising at least one medicament comprising at least one dose of an EZH2 modulator, and at least one medicament comprising at least one dose of an anti-CD19 antibody, said kit for treating cancer further comprising dosing instructions for administering the medicaments for treatment of the patient in recognized need thereof.

In order that this present disclosure be more fully understood, the following examples are set forth. These examples are illustrative only and are not intended to limit the scope of the present disclosure in any way.

### Antibody Sequences

TABLE 1
Tafasitamab (MOR208)

| | SEQ ID NO: | Amino Acids |
|---|---|---|
| HCDR1 | SEQ ID NO: 1 | SYVMH |
| HCDR2 | SEQ ID NO: 2 | NPYNDG |
| HCDR3 | SEQ ID NO: 3 | GTYYYGTRVFDY |
| LCDR1 | SEQ ID NO: 4 | RSSKSLQNVNGNTYLY |
| LCDR2 | SEQ ID NO: 5 | RMSNLNS |
| LCDR3 | SEQ ID NO: 6 | MQHLEYPIT |
| VH | SEQ ID NO: 7 | EVQLVESGGGLVKPGGSLKLSCAASGYTFTSY |
| | | VMHWVRQAPGKGLEWIGYINPYNDGTKYNEK |
| | | FQGRVTISSDKSISTAYMELSSLRSEDTAMYYC |
| | | ARGTYYYGTRVFDYWGQGTLVTVSS |
| VL | SEQ ID NO: 8 | DIVMTQSPATLSLSPGERATLSCRSSKSLQNV |
| | | NGNTYLYWFQQKPGQSPQLLIYRMSNLNSGV |
| | | PDRFSGSGSGTEFTLTISSLEPEDFAVYYCMQ |
| | | HLEYPITFGAGTKLEIK |
| Heavy chain constant | SEQ ID NO: 9 | ASTKGPSVFPLAPSSKSTSGGTAALGCLVKDY |
| domain | | FPEPVTVSWNSGALTSGVHTFPAVLQSSGLYS |
| | | LSSVVTVPSSSLGTQTYICNVNHKPSNTKVDK |
| | | KVEPKSCDKTHTCPPCPAPELLGGPDVFLFPP |
| | | KPKDTLMISRTPEVTCVVVDVSHEDPEVQFNW |
| | | YVDGVEVHNAKTKPREEQFNSTFRVVSVLTVV |
| | | HQDWLNGKEYKCKVSNKALPAPEEKTISKTKG |
| | | QPREPQVYTLPPSREEMTKNQVSLTCLVKGFY |
| | | PSDIAVEWESNGQPENNYKTTPPMLDSDGSF |
| | | FLYSKLTVDKSRWQQGNVFSCSVMHEALHNH |
| | | YTQKSLSLSPGK |
| Light chain constant | SEQ ID NO: 10 | RTVAAPSVFIFPPSDEQLKSGTASVVCLLNNFY |
| domain | | PREAKVQWKVDNALQSGNSQESVTEQDSKD |
| | | STYSLSSTLTLSKADYEKHKVYACEVTHQGLS |
| | | SPVTKSFNRGEC |
| Full Heavy chain | SEQ ID NO: 11 | EVQLVESGGGLVKPGGSLKLSCAASGYTFTSY |
| | | VMHWVRQAPGKGLEWIGYINPYNDGTKYNEK |
| | | FQGRVTISSDKSISTAYMELSSLRSEDTAMYYC |
| | | ARGTYYYGTRVFDYWGQGTLVTVSSASTKGP |
| | | SVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKS |
| | | CDKTHTCPPCPAPELLGGPDVFLFPPKPKDTL |
| | | MISRTPEVTCVVVDVSHEDPEVQFNWYVDGV |
| | | EVHNAKTKPREEQFNSTFRVVSVLTVVHQDW |
| | | LNGKEYKCKVSNKALPAPEEKTISKTKGQPRE |
| | | PQVYTLPPSREEMTKNQVSLTCLVKGFYPSDI |
| | | AVEWESNGQPENNYKTTPPMLDSDGSFFLYS |
| | | KLTVDKSRWQQGNVFSCSVMHEALHNHYTQK |
| | | SLSLSPGK |
| Full Light chain | SEQ ID NO: 12 | DIVMTQSPATLSLSPGERATLSCRSSKSLQNV |
| | | NGNTYLYWFQQKPGQSPQLLIYRMSNLNSGV |
| | | PDRFSGSGSGTEFTLTISSLEPEDFAVYYCMQ |
| | | HLEYPITFGAGTKLEIKRTVAAPSVFIFPPSDEQ |
| | | LKSGTASVVCLLNNFYPREAKVQWKVDNALQ |
| | | SGNSQESVTEQDSKDSTYSLSSTLTLSKADYE |
| | | KHKVYACEVTHQGLSSPVTKSFNRGEC |

### EXAMPLES

### Example 1: Methods In Vitro

### Cell Lines

A panel of 6 lymphoma cell lines was included in the analysis (5 DLBCL and 1 Burkitt lymphoma). All cell lines were cultured in RPMI 1640 medium (Gibco) supplemented with 10%-20% FCS (Sigma) at 37 °C in 5% CO2.

### Compound

Compound 1 was prepared following the methods described in WO 2021/016409, the contents of which are incorporated herein by reference.

### Viability Assay

To analyze direct cytotoxic and anti-proliferative effects on target cells, cell viability assays were performed using the CellTiter-Glo Kit (Promega), a luminescence assay for determination of viable cell number based on quantification of adenosine triphosphate (ATP) levels. Cells were treated with Compound 1 or tazemetostat for 7 (SU-DHL-4, SU-DHL-6, Ramos, U-2932) or 11 days (SU-DHL-8, NU-DUL-1), while tafasitamab was added for the last 1 (SU-DHL-6, NU-DUL-1) or 4 days (all other cell lines) only. Cell densities were adjusted at days 3 and 7 during the assay to avoid overgrowth. The assay was performed in clear 48- and 96-F-well bottom plates in a final volume of 1 mL or 200 μL, respectively, in a cell incubator at 37 °C and 5% CO2. In each experiment, all test conditions were performed in triplicates.

Cell viability was analyzed according to the manufacturer's protocol using the Tecan Infinite F500 plate reader. The relative viable cell count was calculated from the measured relative luminescence units (RLU) with the following equation:

$$\% Relative viable cell count = ( RLU treated cells - RLU background ) / RLU \cdot DMSO - control * 100$$

### CD19 Expression

CD19 expression on the cell surface of SU-DHL-4 and SU-DHL-6 cells was analyzed after 7 days of Compound 1 treatment. Residual cells after CTG readout were stained in duplicates with a live/dead staining kit (L/D fixable yellow stain, Invitrogen) and a PE labeled anti-CD19 detection antibody (clone HIB-19, Biolegend) to measure CD19 expression on the surface of living cells with a flow cytometer (FACS Verse) . . . . Relative CD19 expression was calculated from the measured median fluorescence intensity (MFI) with the following equation:

$$\% Relative CD 19 expression = MFI treated cells / MFI DMSO - control * 100$$

### Data Analysis

For graphical illustration and IC50 determination, the mean values of triplicates (viability assay) or duplicates (CD19 expression) of the individual experiments were imported into GraphPad Prism software (version 8). The mean of mean values−/+standard deviation of relative viable cell count as well as relative CD19 expression were calculated from 2 to 11 individual experiments and displayed as scatter bar plots. IC50 curves were determined applying a non-linear regression curve fit model (sigmoidal dose response, variable slope). The combinatorial effect of both compounds on the cell viability was estimated via a linear mixed effect model of the form

$${Y}_{i , TAFA , Compound 1 , ( Experiment )} = μ + {α}_{TAFA} + {β}_{Compound 1} + {αβ}_{TAFA , Compound 1} + {E}_{Experiment} + {ϵ}_{i , TAFA , Compound 1 , Experiment}$$

with Y_{i TAFA Compound 1 (Experiment)} denoting the log value of the relative luminescence units (RLU) of replicate i of a sample treated with tafasitamab, Compound 1 or the combination of both compounds of an experiment, μ the mean log value of the untreated samples, α_{TAFA} log value of the relative effect of tafasitamab, β_{compound 1} log value of the relative effect of Compound 1, αβ_{TAFA Compound 1} the relative combination effect of tafasitamab and Compound 1 together, E_{Experiment} the relative random effect of the experiment and ϵ_{i, TAFA, Compound 1, Experiment} the residuum of replicate i of a sample treated with tafasitamab, Compound 1 or the combination of both compounds of an experiment.

The grand mean RLU values were calculated for.
untreated samples via e^{μ}
tafasitamab treated samples via e^{(μ+α)}
Compound 1 treated samples via e^{(μ+β)}
tafasitamab+Compound 1 treated samples via e^{(μ+α+β+αβ)}

The percent inhibition of tafasitamab, Compound 1 as well as the combination effect of both compounds were calculated for
tafasitamab via (e^{α}−1)*−100
Compound 1 via (e^{β}−1)*−100.
combination effect via (e^{αβ}−1)*−100

### Results In Vitro

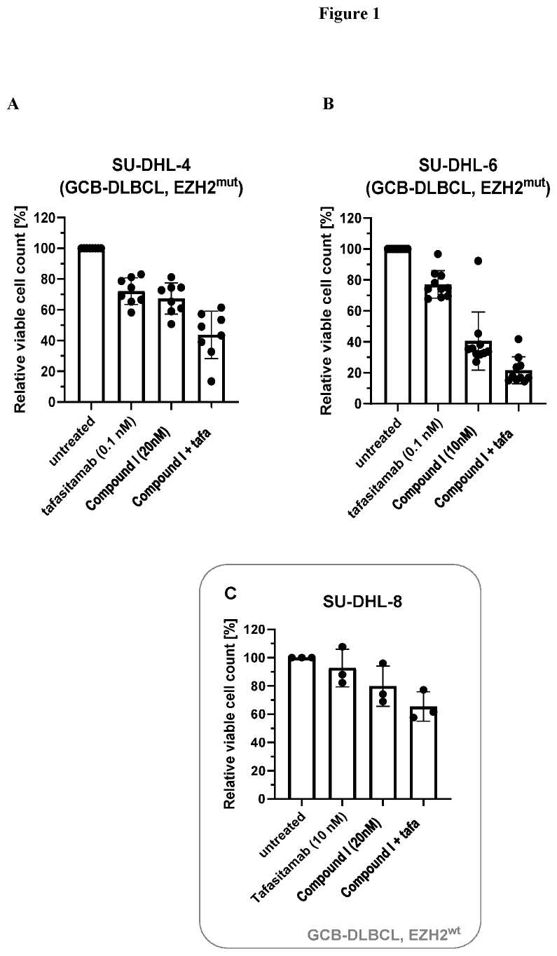

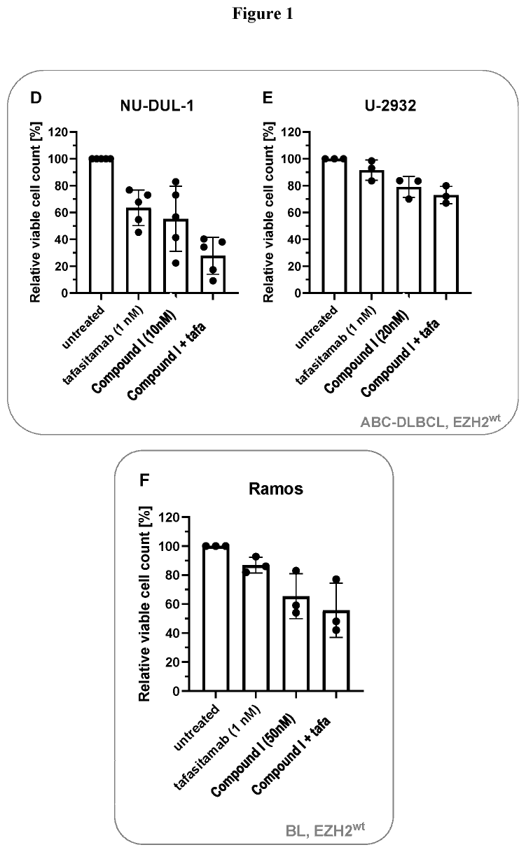

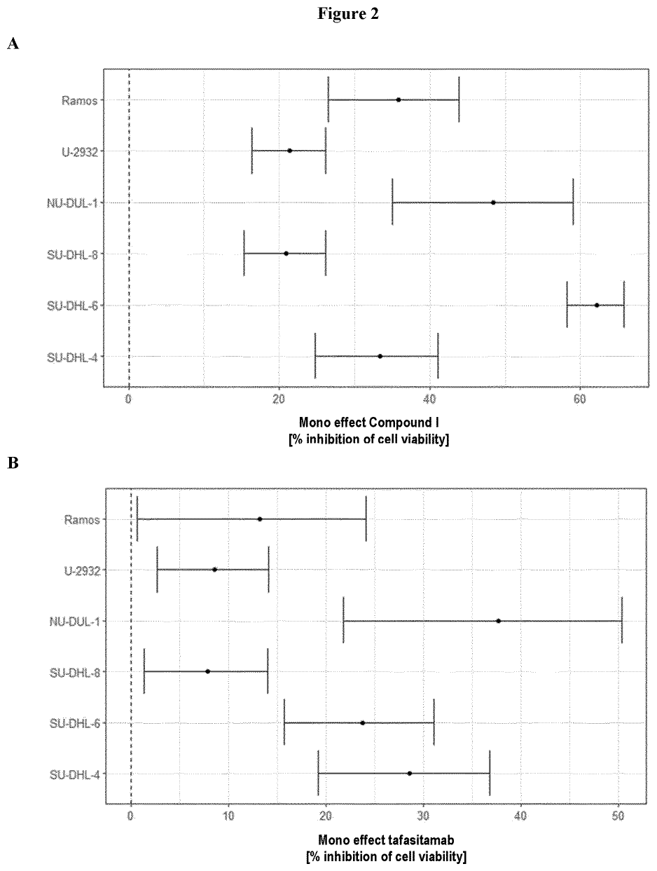

Tafasitamab mediates B-cell lysis through direct cytotoxic effects and immune effector mechanisms like ADCC and ADCP, while Compound 1 has cytotoxic and anti-proliferative effects on tumor cells. The combinatorial effect of both drugs was analyzed for their shared mode-of-action, i.e. their direct effects on the number of viable target cells in a sample. Lymphoma cell lines used for in vitro experiments comprised cell lines from different NHL subtypes (ABC type DLBCL, GCB type DLBCL, and BL) and with different EHZ2 status (wildtype and variants with gain-of-function mutation) and differ in their sensitivity to The effect of tafasitamab, Compound 1, and the combination of both on the tumor cell viability at pre-determined suboptimal dose levels is shown in FIG. 1. In 7 out of 7 tested cell lines, the compound combination lead to a better reduction of the relative viable cell count than the individual compounds. The combinatorial effect of tafasitamab and Compound 1 was estimated via the linear mixed effect model. Estimated combinatorial effects below 0 indicate an antagonistic effect, 0 indicates an additive effect, and values above 0 indicate an additional effect above the sum of individual effects (synergistic effect). The individual effect of both compounds estimated by the applied model (FIGS. 2A and B) correlated well with the results shown in FIG. 1. Evaluation of the combinatorial effect resulted in significant synergistic reduction of viability of SU-DHL-4, SU-DHL-6 and SU-DHL-8, a trend to reduce synergistically the viability of NU-DUL-1, and additive but not synergistic reduction of viability of U-2932 and Ramos cells. (FIG. 2C).

Tafasitamab's ADCC mode-of-action was not negatively influenced by Compound 1, when effector cells had been pre-treated with Compound 1 (data not shown).

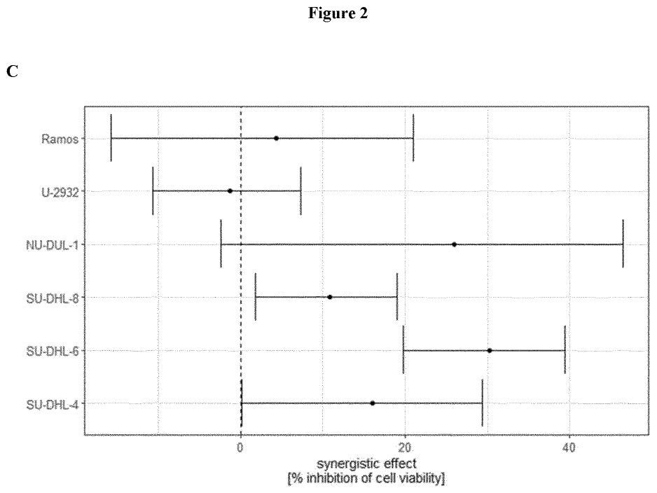

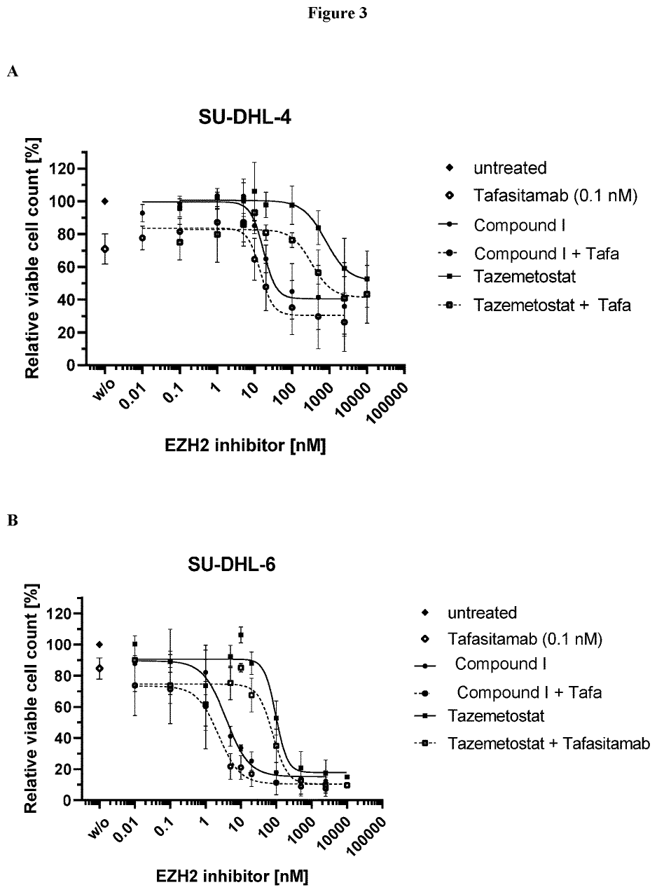

The effect of Compound 1 alone and in combination with tafasitamab was compared to another EZH2 inhibitor, tazemetostat. In both tested cell lines, SU-DHL-4 and SU-DHL-6, Compound 1 inhibited viability at lower concentrations compared to tazemetostat. The half maximal inhibitory concentration (IC50) for Compound 1/tafasitamab combination was 23-fold (14.3 nM vs 332 nM in SU-DHL-4) or 35-fold (2.3 nM vs 79.4 nM in SU-DHL-6) improved compared to tazemetostat/tafasitamab combination. Overall, Tafasitamab can improve activity of EZH2 inhibitors in general as shown by its combinatorial effect with tazemetostat in CTG assay with SU-DHL-4 and SU-DHL-6 cells (FIGS. 3A and 3B, Table 1).

TABLE 1

| IC50 | | | | |
|---|---|---|---|---|
| in nM | | Compound 1 + | | tazemetostat + |
| [95% CI] | Compound 1 | tafasitamab | tazemetostat | tafasitamab |
| SU- | 17.1 | 14.3 | 792 | 332 |
| DHL-4 | [12.0 to | [9.1 to 31.5] | [291 to | [90.0 to 3919] |
| | 32.3] | | 114321943] | |
| SU- | 3.8 | 2.3 | 97.4 | 79.4 |
| DHL-6 | [2.2 to 6.1] | [1.1 to 4.5] | [60.1 to 177] | [39.4 to 152.6] |

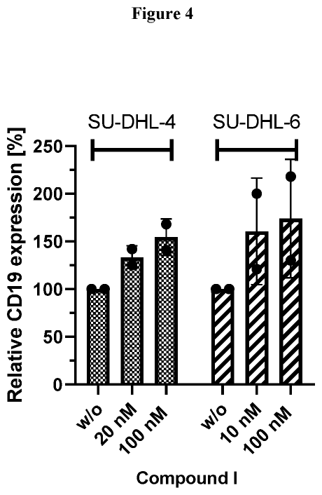

In parallel to the viability read-out, Compound 1 treated SU-DHL-4 and SU-DHL-6 cells were analyzed for their expression of the tafasitamab target molecule CD19. As determined by flow cytometric analysis after 7 days of treatment, Compound 1 led to an increase of the CD19 surface level which could be an explanation for improved cytotoxic effects by tafasitamab (FIG. 4).

### Example 2: Methods In Vivo

### Methods

The in vivo study was carried out in WSU-DLCL2 tumor bearing CB17-SCID mice. 4×10\hat{ }6 WSU-DLCL2 cells (DLBCL cell line) per animal were implanted with matrigel (1:1) s.c. into the left flank of 6-8 weeks old female CB17-SCID mice. On study day 17, when a mean tumor volume of approximately 100 mm\hat{ }3 was reached, animals were randomly allocated into treatment groups with 8 mice per group and treatment was started. Treatment was applied every day p.o. at 35 mg/kg or 50 mg/kg for Compound 1 (application volume: 10 mL/kg; formulation buffer: 20% propylene glycol, 10% Solutol HS15, 70% phthalate buffer) and biweekly i.v. at 3 mg/kg or 10 mg/kg for tafasitamab (application volume: 5 mL/kg; formulation buffer: PBS). Tumor volume and body weight were measured twice weekly. Mice were sacrificed when reaching a tumor volume >1500 mm\hat{ }3 or latest at study end on day 69 after tumor cell implantation. Mean tumor growth curves were calculated using GraphPad Prism software (version 8.4.3).

### Results

The proof of concept in vivo study was performed in WSU-DLCL2 tumor bearing CB17-SCID mice. Table 2 summarizes the nine different treatment groups.

TABLE 2

| | Animals | | Treatment | Dosing | | | |
|---|---|---|---|---|---|---|---|
| | per | Start of | Compound I | Dosis | | Volume | |
| Group | group | treatment | formulation buffer | [mg/kg] | Regimen | [mL/kg] | Route |
| 1 | 8 | at ~100 mm3 | Tafasitamab | 0 | QD | 10 | p.o. |
| | | | formulation buffer | 0 | BIW | 5 | i.v. |
| 2 | 8 | at ~100 mm3 | Compound I high | 50 | QD | 10 | p.o. |
| 3 | 8 | at ~100 mm3 | Compound I low | 35 | QD | 10 | p.o. |
| 4 | 8 | at ~100 mm3 | Tafasitamab high | 10 | BIW | 5 | i.v. |
| 5 | 8 | at ~100 mm3 | Tafasitamab low | 3 | BIW | 5 | i.v. |
| 6 | 8 | at ~100 mm3 | Compound I high | 50 | QD | 10 | p.o. |
| | | | Tafasitamab high | 10 | BIW | 5 | i.v. |
| 7 | 8 | at ~100 mm3 | Compound I high | 50 | QD | 10 | p.o |
| | | | Tafasitamab low | 3 | BIW | 5 | i.v. |
| 8 | 8 | at ~100 mm3 | Compound I low | 35 | QD | 10 | p.o. |
| | | | Tafasitamab high | 10 | BIW | 5 | i.v. |
| 9 | 8 | at ~100 mm3 | Compound I low | 35 | QD | 10 | 0.0. |
| | | | Tafasitamab low | 3 | BIW | 5 | i.v. |

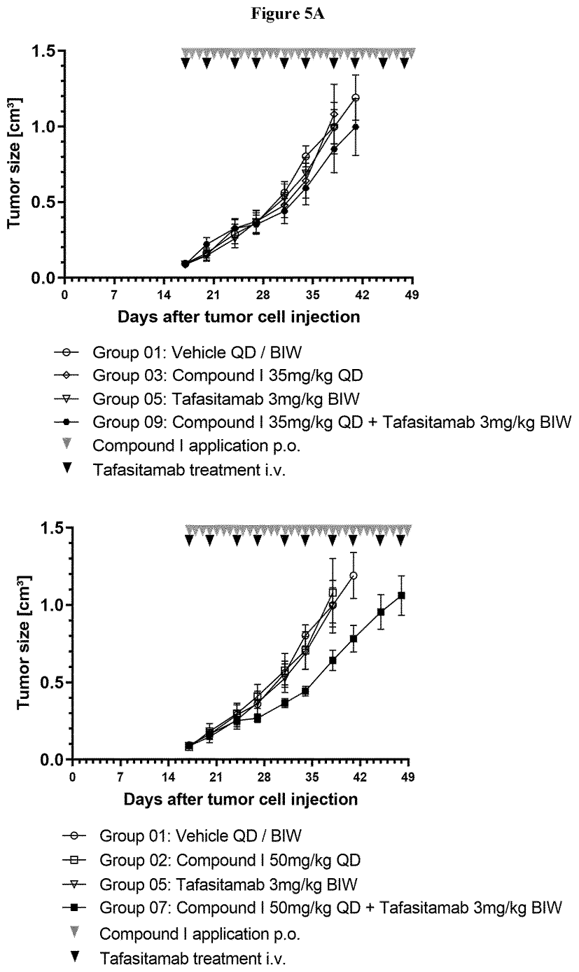

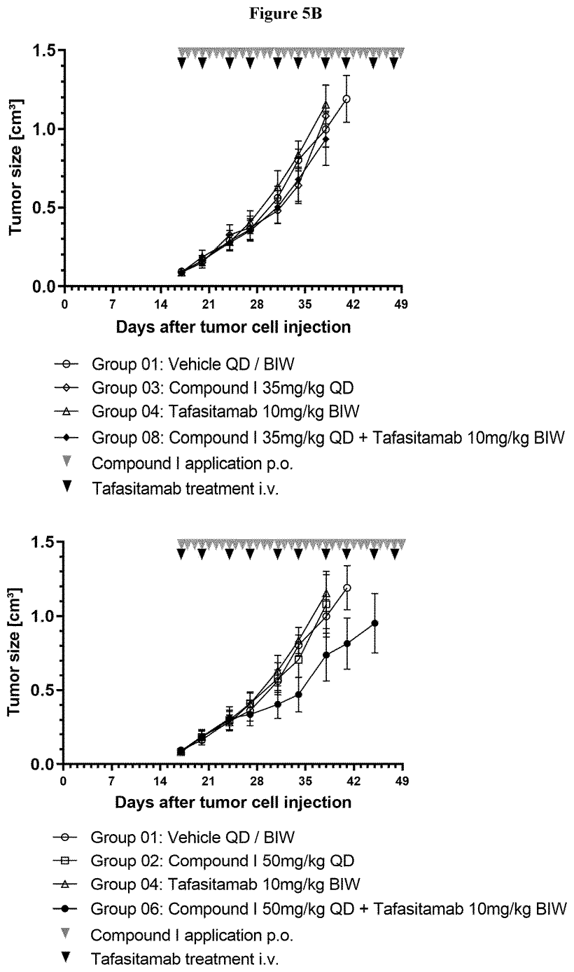

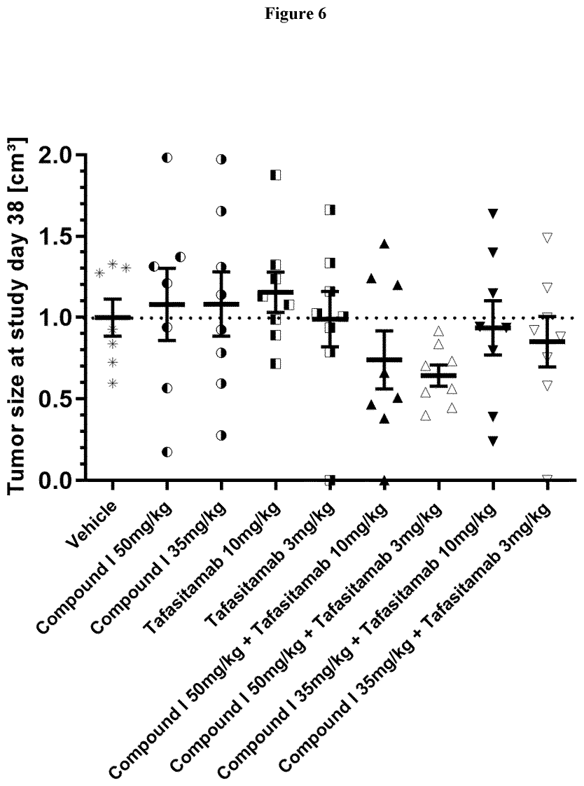

FIGS. 5A and 5B show the tumor growth kinetics (mean±/−SEM) of the performed in vivo model. Mean tumor volumes of the individual treatment groups on day 38 after tumor implantation (day 21 after treatment start) are depicted in FIG. 6.

Monotherapies with Compound 1 (50 and 35 mg/kg) or tafasitamab (10 and 3 mg/kg) as well as combination treatments of 35 mg/kg Compound 1 with any of the two tested tafasitamab doses did not reveal any tumor growth inhibition in this model. However, treatment with the higher Compound 1 dose (50 mg/kg) plus tafasitamab at both doses (10 and 3 mg/kg) showed a combinatorial effect. 50 mg/kg Compound 1 in combination with 10 mg/kg tafasitamab led to a tumor growth inhibition of 26% and 50 mg/kg Compound 1 in combination with 3 mg/kg tafasitamab led to a tumor growth inhibition of 36%, compared to vehicle treated control group. Body weights progressed normally for all animals in this study (data not shown).

What is claimed is:

1. A method of treating a cancer, comprising administering to a human subject in need of said treatment a combination of an EZH2 modulator and an anti-CD19 antibody.

2. The method of claim 1, wherein the EZH2 modulator is of the chemical formula: 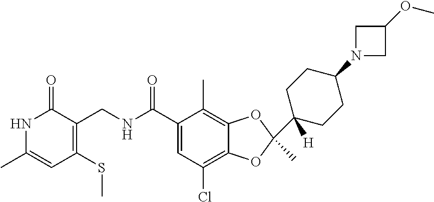
or a pharmaceutically acceptable salt thereof.

3. The method of any one of claims 1-2, wherein the anti-CD19 antibody comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising the sequence LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

4. The method of any one of claims 1-3, wherein the anti-CD19 antibody comprises a heavy chain variable region of
(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS
and
a light chain variable region of
(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

5. The method of any one of claims 1-5, wherein the anti-CD19 antibody comprises an Fc domain comprising an amino acid substitution at position S239 and/or 1332, wherein the numbering is according to the EU index as in Kabat.

6. The method of any one of claims 1-4, wherein the anti-CD19 antibody comprises an Fc domain comprising an S239D amino acid substitution and an I332E amino acid substitution, wherein the numbering is according to the EU index as in Kabat.

7. The method of any one of claims 1-6, wherein the anti-CD19 antibody comprises a heavy chain region of
(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K
and
a light chain region of
(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

8. The method of any one of claims 1-7, wherein the EZH2 modulator, is administered orally.

9. The method of any one of claims 1-8, wherein the EZH2 modulator, is administered intravenously, or subcutaneously.

10. The method of any one of claims 1-9, wherein the EZH2 modulator, is administered by intravenous infusion.

11. The method of any one of claims 1-10, wherein the cancer is a CD19 positive cancer.

12. The method of any one of claims 1-11, wherein the cancer is a hematological malignancy.

13. The method of any one of claims 1-12, wherein the cancer is a lymphoma or a leukemia.

14. The method of any one of claims 1-13, wherein the cancer is chronic lymphocytic leukemia, acute lymphoblastic leukemia, or a non-Hodgkin's lymphoma.

15. The method of any one of claims 1-14, wherein the cancer is a non-Hodgkin's lymphoma selected from the group consisting of follicular lymphoma (FL), small lymphocytic lymphoma, mucosa-associated lymphoid tissue lymphoma, marginal zone lymphoma (MZL), mantle cell lymphoma (MCL), diffuse large B-cell lymphoma (DLBCL), and Burkitt lymphoma.
